(19) United States
(12) Patent Application Publication
(10) Pub. No.: US 2007/0282432 A1
(43) Pub. Date: Dec. 6, 2007

(54) Implantable medical endoprostheses

(76) Inventors: Jonathan S. Stinson, Plymouth, MN (US); Matthew J. Miller, Stillwater, MN (US)

Correspondence Address: FISH & RICHARDSON PC, P.O. BOX 1022, MINNEAPOLIS, MN, 55440-1022, US

(21) Appl. No.: 11/443,942

(22) Filed: May 31, 2006

(51) Int. Cl. A61F 2/02; A61F 2/82

(52) U.S. Cl. 623/1.38; 623/1.46; 623/23.75; 427/2.24; 623/1.39; 148/519; 148/902

(57) ABSTRACT

Implantable medical endoprostheses, such as stents, are disclosed.

### TECHNICAL FIELD

This disclosure relates to implantable medical endoprostheses, and related systems and methods.

### BACKGROUND

Implantable medical endoprostheses can be placed in a lumen in the body. Examples of implantable medical endoprostheses include stents (e.g., covered stents and stent-grafts).

### SUMMARY

This disclosure relates to implantable medical endoprostheses, and related systems and methods.

In one aspect, the invention generally features an implantable medical endoprosthesis that includes a material. The implantable medical endoprosthesis has a first region and a second region. In the first region the material is in a first solid phase. In the second region the material is in a second solid phase different from the first solid phase.

In another aspect, the invention generally features a method of making an implantable medical endoprosthesis. The method includes heating a region of an implantable medical endoprosthesis. The implantable medical endoprosthesis includes a material, and heating the region of the implantable medical endoprosthesis converts the material from a first solid phase to a second solid phase different from the first solid phase. The method further includes cooling the heated region under conditions that allow the material in the heated region to remain in the second phase.

In an additional aspect, the invention generally features an implantable medical endoprosthesis having a first region and a second region. The first region includes a first material, and the second region includes the first material coated with a second material, where the second material is selected to increase an erosion rate of the second region with respect to the first region in a body lumen.

In a further aspect, the invention generally features an implantable medical endoprosthesis having a first region and a second region. The first and second regions include a material, and the second region has pores.

In one aspect, the invention generally features a method of making an implantable medical endoprosthesis. The method includes heating a region of an implantable medical endoprosthesis. The implantable medical endoprosthesis includes a material. The region of the implantable medical endoprosthesis is heated to a temperature greater than a melting temperature of the material. The method also includes disposing gas through the heated region, and cooling the heated region so that at least some of the gas is trapped in the heated region.

In another aspect, the invention generally features a method of making an implantable medical endoprosthesis. The method includes coating surfaces of a first region of an implantable medical endoprosthesis with a masking agent, contacting a saline solution to be in contact with one or more surfaces of a second region of an implantable medical endoprosthesis, directing an electric current to flow through the saline solution, and removing the masking agent.

In an additional aspect, the invention generally features an implantable medical endoprosthesis having inner and outer surfaces that define a wall that extends along a longitudinal axis of the implantable medical endoprosthesis. A first region of the wall has a first thickness in a direction transverse to the longitudinal axis, and a second region of the wall has a second thickness in the same direction that is less than the first thickness. The first and second regions include a material, and the second region has pores.

In a further aspect, the invention generally features an implantable medical endoprosthesis including a material. The implantable medical endoprosthesis has a first region and a second region. In the first region, the material is in a first solid phase. In the second region, the material is in a second solid phase different from the first solid phase.

In one aspect, the invention generally a method of making an implantable medical endoprosthesis. The method includes heating a region of an implantable medical endoprosthesis. The implantable medical endoprosthesis includes a material. Heating the region of the implantable medical endoprosthesis converts the material from a first solid phase to a second solid phase different from the first solid phase. The method also includes cooling the heated region under conditions that allow the material in the heated region to remain in the second phase.

In another aspect, the invention generally features an implantable medical endoprosthesis having first and second regions. The first region includes a first material, and the second region includes the first material coated with a second material. The second material is selected to increase an erosion rate of the second region with respect to the first region in a body lumen.

Embodiments can include one or more of the following advantages.

In some embodiments, an endoprosthesis can erode over time in a body lumen, allowing the lumen to return to a natural condition without an endoprosthesis present.

In certain embodiments, erosion of an endoprosthesis over time in a body lumen can reduce effects that arise from introducing a foreign object into the body lumen.

In some embodiments, an endoprosthesis can have surface features that lead to a controlled fragmentation of an endoprosthesis in a body lumen. In certain embodiments, controlled fragmentation of the endoprosthesis can include producing endoprosthesis fragments having selected lengths. In certain embodiments, controlled fragmentation of the endoprosthesis can include producing fragments in a selected order.

In some embodiments, an endoprosthesis can have surface features to select an average erosion rate of particular regions of the endoprosthesis to determine an average erosion time of the endoprosthesis regions in a body lumen.

In certain embodiments, an endoprosthesis can have surface features to reduce the mechanical strength of the endoprosthesis in particular regions of the endoprosthesis structure.

In some embodiments, the ratio of a length of certain surface features to a depth of the same features can be selected to enhance a corrosion rate of an endoprosthesis wall adjacent to the features due to crevice corrosion effects.

Other features and advantages of the invention will be apparent from the description, drawings, and claims.

### DESCRIPTION OF DRAWINGS

FIG. 1A is a perspective view of an embodiment of an implantable medical endoprosthesis.

FIG. 1B is a cross-sectional view of the implantable medical endoprosthesis of FIG. 1A taken along line 1B-1B.

FIG. 2 is a cross-sectional view of an embodiment of an implantable medical endoprosthesis.

FIG. 3 is a cross-sectional view of an embodiment of an implantable medical endoprosthesis.

FIG. 4 is a cross-sectional view of an embodiment of an implantable medical endoprosthesis.

FIG. 5 is a cross-sectional view of an embodiment of an implantable medical endoprosthesis.

FIG. 6 is a cross-sectional view of an embodiment of an implantable medical endoprosthesis.

FIG. 7 is a cross-sectional view of an embodiment of an implantable medical endoprosthesis.

FIG. 8 is a perspective view of an embodiment of an implantable medical endoprosthesis.

FIG. 9A is a perspective view of an embodiment of an implantable medical endoprosthesis.

FIG. 9B is a cross-sectional view of the implantable medical endoprosthesis of FIG. 9A taken along line 9B-9B.

FIG. 10A is a perspective view of an embodiment of an implantable medical 20 endoprosthesis.

FIG. 10B is a cross-sectional view of the implantable medical endoprosthesis of FIG. 10A taken along line 10B-10B.

FIG. 11 is a cross-sectional view of an embodiment of an implantable medical endoprosthesis.

FIG. 12 is a cross-sectional view of an embodiment of an implantable medical endoprosthesis.

FIG. 13A is a perspective view of an embodiment of an implantable medical endoprosthesis.

FIG. 13B is a cross-sectional view of the implantable medical endoprosthesis of FIG. 13A taken along line 13B-13B.

FIG. 14 is a cross-sectional view of an embodiment of an implantable medical endoprosthesis.

FIG. 15 is a cross-sectional view of an embodiment of an implantable medical endoprosthesis.

FIG. 16 is a cross-sectional view of an embodiment of an implantable medical endoprosthesis.

FIGS. 17-19 are side views of an embodiment of an endoprosthesis delivery system during use.

Like reference symbols in the various drawings indicate like elements.

### DETAILED DESCRIPTION

The disclosure relates to implantable medical endoprostheses that can have structural, compositional, and/or other features designed to control a rate of degradation of the endoprosthesis within a body lumen, and/or to control the morphologies of the fragments resulting from degradation. In some embodiments, an implantable medical endoprosthesis can be a stent (e.g., a self-expanding stent, a balloon-expandable stent). Examples of stents include coronary stents, aortic stents, peripheral vascular stents, gastrointestinal stents, urology stents and neurology stents.

### Structural and Mechanical Features

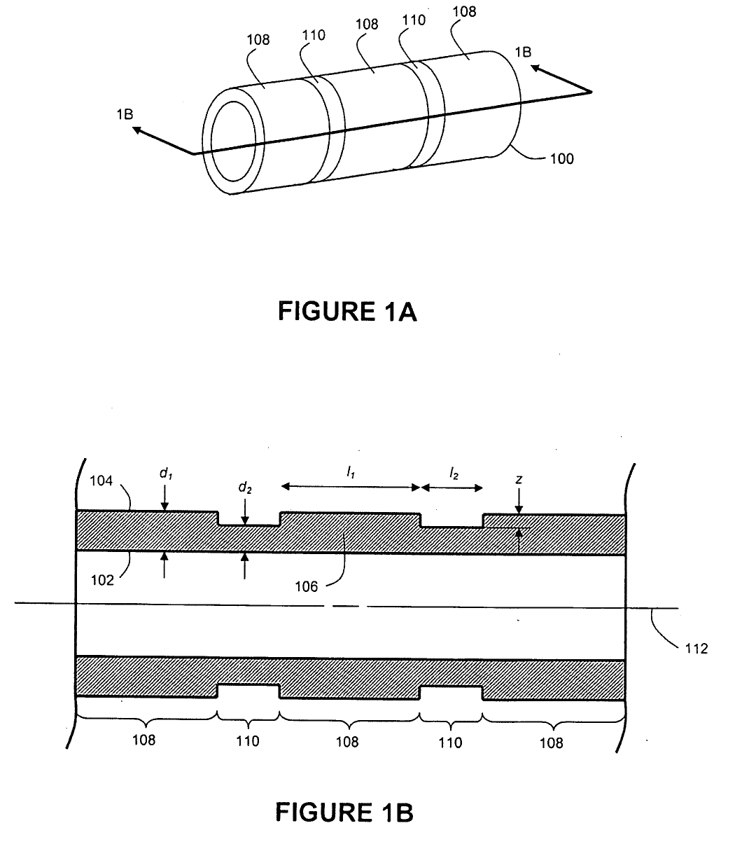

FIGS. 1A and 1B show perspective and cross-sectional views, respectively, of a stent 100 having structural features that influence a stent erosion rate and fragmentation pattern. Stent 100 is tubular and has an inner surface 102 and an outer surface 104. The material between these surfaces forms a stent wall 106. Stent wall 106 has surface features which are provided to control fragmentation of stent 100 within a body lumen. First regions 108 of stent 100 have a thickness in a radial direction (transverse to a longitudinal axis 112 of stent 100) of d₁. Second regions 110 of stent 100 have a thickness in a radial direction (transverse to longitudinal axis 112) d₂ that is less than d₁.

Generally, the properties of stent 100, including the number and cross-sectional shapes of regions 108 and 110, and the thicknesses and lengths of regions 108 and 110, as well as the material(s) from which stent 100 is formed, are selected to provide desired erosion and/or fragmentation characteristics (e.g., an average time before erosion leads to mechanical failure of stent 100, an average time before erosion leads to formation of one or more fragments from stent 100, an average size of fragments formed by erosion of stent 100). As an example, in some embodiments in which stent 100 is a coronary stent, the thickness of regions 110 is chosen so that erosion of stent wall 106 in at least some of regions 110 is complete in a time from 3 months to 6 months following placement of stent 100 into a coronary lumen. As another example, in certain embodiments in which stent 100 is a tracheal stent, the thickness of regions 110 can be chosen so that erosion of stent wall 106 in at least some of regions 110 is complete in a time from 6 months to 24 months following implantation of stent 100 in a tracheal lumen.

In general, when disposed in a body lumen, erosion of stent 100 occurs both in first regions 108 and in second regions 110 at the same time and at the same rate, because stent wall 106 is formed from a single material. However, when stent 100 erodes within a body lumen, erosion through stent wall 106 is typically complete in second regions 110 before it is complete in first regions 108 because d₂ is less than d₁.

Generally, a length l₁, of first regions 108 in a direction of axis 112 of stent 100 can be selected as desired. The length of regions 108 can be selected, for example, to control a length of stent fragments resulting from erosion of stent 100 within a body lumen. Because erosion is typically complete in regions 110 before it is complete in regions 108, the length of regions 108 approximately determines the length of fragments of stent 100. For example, in some embodiments, length l₁ is 1 micron or more (e.g., 2 microns or more, 5 microns or more, 10 microns or more, 20 microns or more, 50 microns or more, 100 microns or more, 250 microns or more, 500 microns or more, 1 millimeter or more, 2 millimeters or more, 5 millimeters or more, 10 millimeters or more), and/or length l₁ is 50 millimeters or less (e.g., 40 millimeters or less, 30 millimeters or less, 20 millimeters or less, 10 millimeters or less, 5 millimeters or less, 2 millimeters or less, 1 millimeter or less, 500 microns or less, 250 microns or less, 100 microns or less, 50 microns or less, 40 microns or less, 30 microns or less, 20 microns or less, 10 microns or less).

In general, a length l₂ of second regions 110 in a direction of axis 112 of stent 100 can be selected as desired to provide larger or smaller regions of stent 100 in which erosion of stent wall 30 106 is complete in a shorter time than erosion of stent wall 106 in regions 108. For example, in certain embodiments, length l₂ is 10 millimeters or less (e.g., 8 millimeters or less, 6 millimeters or less, 4 millimeters or less, 2 millimeters or less, 1 millimeter or less, 750 microns or less, 500 microns or less, 250 microns or less, 150 microns or less, 100 microns or less, 50 microns or less, 20 microns or less, 10 microns or less, 5 microns or less, 2 microns or less, 1 micron or less), and/or length l₂ is 1 micron or more (e.g., 5 microns or more, 10 microns or more, 20 microns or more, 50 microns or more, 100 microns or more, 150 microns or more, 250 microns or more, 500 microns or more 750 microns or more, 1 millimeter or more, 2 millimeters or more, 4 millimeters or more, 6 millimeters or more, 8 millimeters or more, 10 millimeters or more).

The thickness d₁ of regions 108 can generally be selected as desired. For example, the thickness d₁ of regions 108 can be selected to impart desired mechanical properties to stent 100. In some embodiments, for example, d₁ can be 20 microns or more (e.g., 50 microns or more, 100 microns or more, 150 microns or more, 200 microns or more, 300 microns or more, 400 microns or more, 500 microns or more, 750 microns or more, 1 millimeter or more, 1.5 millimeters or more, 2 millimeters or more, 3 millimeters or more, 5 millimeters or more), and/or d₁ can be 10 millimeters or less (e.g., 5 millimeters or less, 3 millimeters or less, 2 millimeters or less, 1.5 millimeters or less, 1 millimeter or less, 750 microns or less, 500 microns or less, 400 microns or less, 300 microns or less, 200 microns or less, 150 microns or less, 100 microns or less, 50 microns or less).

For a selected material forming stent wall 106, the thickness d₂ of regions 110 can be selected to provide a desired average erosion time of stent 100 in a body lumen. In certain embodiments, the thickness d₂ can be 10 millimeters or less (e.g., 5 millimeters or less, 3 millimeters or less, 2 millimeters or less, 1.5 millimeters or less, 1 millimeter or less, 750 microns or less, 500 microns or less, 400 microns or less, 300 microns or less, 200 microns or less, 150 microns or less, 100 microns or less, 50 microns or less). Alternatively, or in addition, in certain embodiments, the thickness d₂ can be 20 microns or more (e.g., 50 microns or more, 100 microns or more, 150 microns or more, 200 microns or more, 300 microns or more, 400 microns or more, 500 microns or more, 750 microns or more, 1 millimeter or more, 1.5 millimeters or more, 2 millimeters or more, 3 millimeters or more, 5 millimeters or more).

In some embodiments, the thickness d₂ of regions 110 can be 10% or more (e.g., 20% or more, 25% or more, 30% or more, 50% or more, 60% or more, 75% or more, 85% or more, 90% or more, 95% or more, 98% or more) of the thickness d₁ of regions 108.

In some embodiments, a ratio of l₁ to d₁ in regions 108 can be 0.01 or more (e.g., 0.02 or more, 0.05 or more, 0.1 or more, 0.5 or more, 1 or more, 10 or more, 50 or more, 100 or more). Alternatively, or in addition, the ratio of l₁ to d₁ in regions 108 can be 1000 or less (e.g., 100 or less, 50 or less, 10 or less, 5 or less, 1 or less, 0.5 or less, 0.1 or less, 0.05 or less).

In some embodiments, a ratio of l₂ to d₂ in regions 110 can be 0.01 or more (e.g., 0.02 or more, 0.05 or more, 0.1 or more, 0.5 or more, 1 or more, 10 or more, 50 or more, 100 or more). Alternatively, or in addition, the ratio of l₂ to d₂ in regions 110 can be 1000 or less (e.g., 100 or less, 50 or less, 10 or less, 5 or less, 1 or less, 0.5 or less, 0.1 or less, 0.05 or less).

In some embodiments, a ratio of l₁ to l₂ can be 0.01 or more (e.g., 0.02 or more, 0.05 or more, 0.1 or more, 0.5 or more, 1 or more, 10 or more, 50 or more, 100 or more). Alternatively, or in addition, the ratio of l₁ to l₂ can be 1000 or less (e.g., 100 or less, 50 or less, 10 or less, 5 or less, 1 or less, 0.5 or less, 0.1 or less, 0.05 or less).

A difference in thickness between regions 108 and 110 can be defined as z=d₁−d₂, and in some embodiments, a ratio of l₂ to z can be 100 or less (e.g., 50 or less, 10 or less, 1 or less, 0.7 or less, 0.5 or less, 0.3 or less, 0.1 or less, 0.005 or less). Alternatively, or in addition, the ratio of l₂ to z can be 0.001 or more (e.g., 0.005 or more, 0.1 or more, 0.3 or more, 0.5 or more, 0.7 or more, 1 or more, 10 or more, 50 or more). In some embodiments, a ratio of l₂ to z can be selected in order to effectively concentrate mechanical stresses in regions 110. Mechanical stresses can arise from both physiological static and cyclic loading within a body lumen. Regions 110 having smaller ratios of l₂ to z can undergo mechanical failure relatively early after implantation of stent 100 within a body lumen due to concentration of physiological stresses and erosion in regions 110.

The material from which stent 100 is formed can generally be selected as desired. Typically, stent 100 is formed of a material that is biocompatible.

In some embodiments, stent 100 can be formed from a material that contains a metal, such as magnesium, iron, or bismuth. In certain embodiments, stent 100 is formed of an alloy containing more than one metal. Examples of alloys include magnesium alloys (e.g., containing iron and/or bismuth), iron alloys (e.g., low-carbon steel (AISI 1018-1025), medium carbon steel (AISI 1030-1055), and high carbon steel (1060-1095)) and binary bismuth-iron alloys. In some embodiments, stent 100 can be formed from a shape memory material that contains one or more metals. An example of such a material is iron-manganese (Fe—Mn). Metal-containing shape memory materials are disclosed, for example, in Schetsky, L. McDonald, “Shape Memory Alloys”, Encyclopedia of Chemical Technology (3rd Ed.), John Wiley & Sons, 1982, vol. 20, pp. 726-736.

In certain embodiments, stent 100 can be formed from a polymer material (e.g., a biocompatible polymer material). Examples of polymer materials include polylactic acid, polyvinyl acid, polyglycolic acid, polyglycolide lactide, polyphosphates, polyphosphonates, polyphosphoesters, polycapromide and tyrosine-derived polycarbonates. Examples of polymer materials are disclosed in U.S. Pat. No. 6,719,934, which is hereby incorporated by reference. In certain embodiments, stent 100 can be formed of a polymer material that is a shape memory polymer material. Examples of shape memory polymer materials include shape memory polyurethanes (available from Mitsubishi), polynorbomene (e.g., Norsorex™ (Mitsubishi)), polymethylmethacrylate (PMMA), poly(vinyl chloride), polyethylene (e.g., crystalline polyethylene), polyisopropene (e.g., trans-polyisoprene), styrene-butadiene copolymer, and rubbers. Shape memory polymer materials are commercially available from, for example, MnemoScience GmbH (Pauwelsstrasse 19, D-52074 Aachen, Germany).

Although described as being formed of a single material, in some embodiments stent 100 can be formed from more than one material. For example, regions 108 can be formed from a first material having a first erosion rate, and regions 110 can be formed from a different material. The erosion rates of the different materials may be the same, or they may be different. In some embodiments, the erosion rate of 110 can be greater than the erosion rate of 108. In certain embodiments, the erosion rate of 110 can be less than the erosion rate of 108.

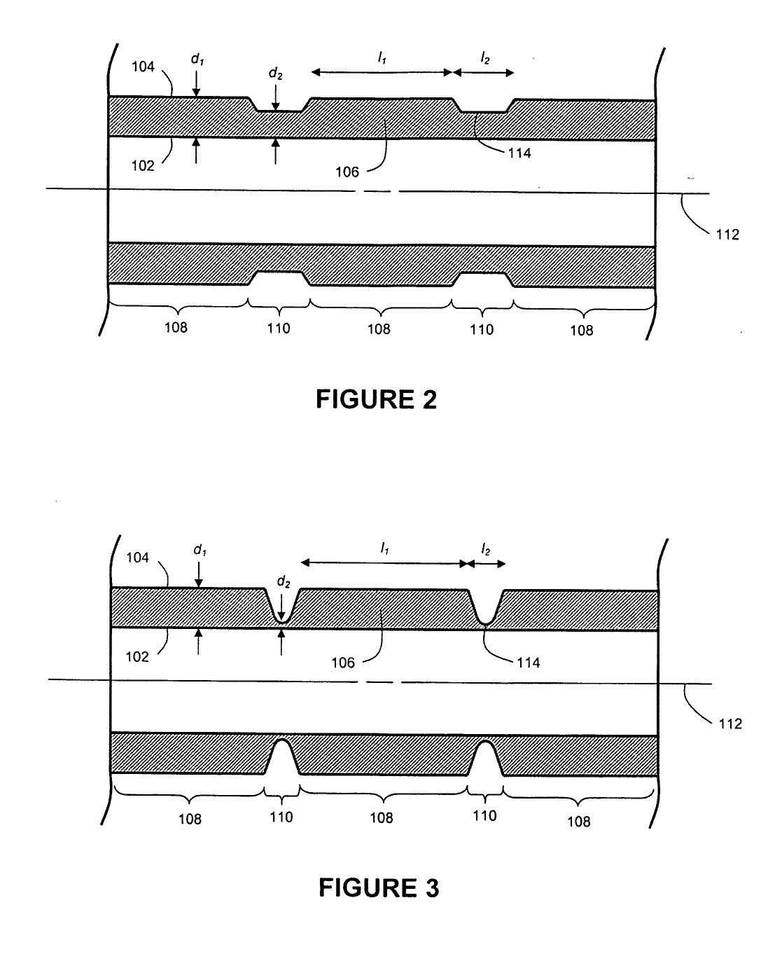

In some embodiments, a cross-sectional shape of regions 110 can be selected to provide stent 100 having desired mechanical and erosion properties. For example, as shown in FIG. 1B, second regions 110 have a rectangular cross-sectional shape. Other cross-sectional shapes are also possible. As an example, FIG. 2 shows an embodiment in which regions 110 have a trapezoidal cross-sectional profile with flat surfaces 114. As another example, FIG. 3 shows an embodiment in which regions 110 have curved surfaces 114.

In embodiments in which regions 110 has curved surfaces, one or more of the curved surfaces can have a radius of curvature R of 0.001 inch or more (e.g., 0.002 inch or more, 0.003 inch or more, 0.004 inch or more, 0.005 inch or more, 0.006 inch or more, 0.007 inch or more, 0.008 inch or more, 0.009 inch or more, 0.01 inch or more). In some embodiments, R can be 0.02 inch or less (e.g., 0.01 inch or less, 0.009 inch or less, 0.008 inch or less, 0.007 inch or less, 0.006 inch or less, 0.005 inch or less, 0.004 inch or less, 0.003 inch or less, 0.002 inch or less, 0.001 inch or less).

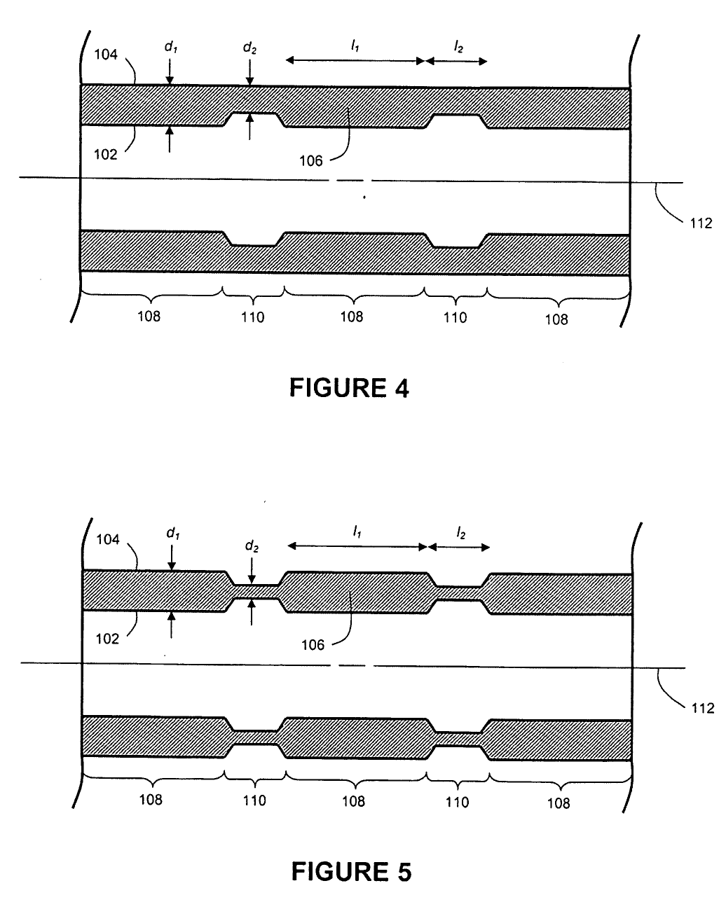

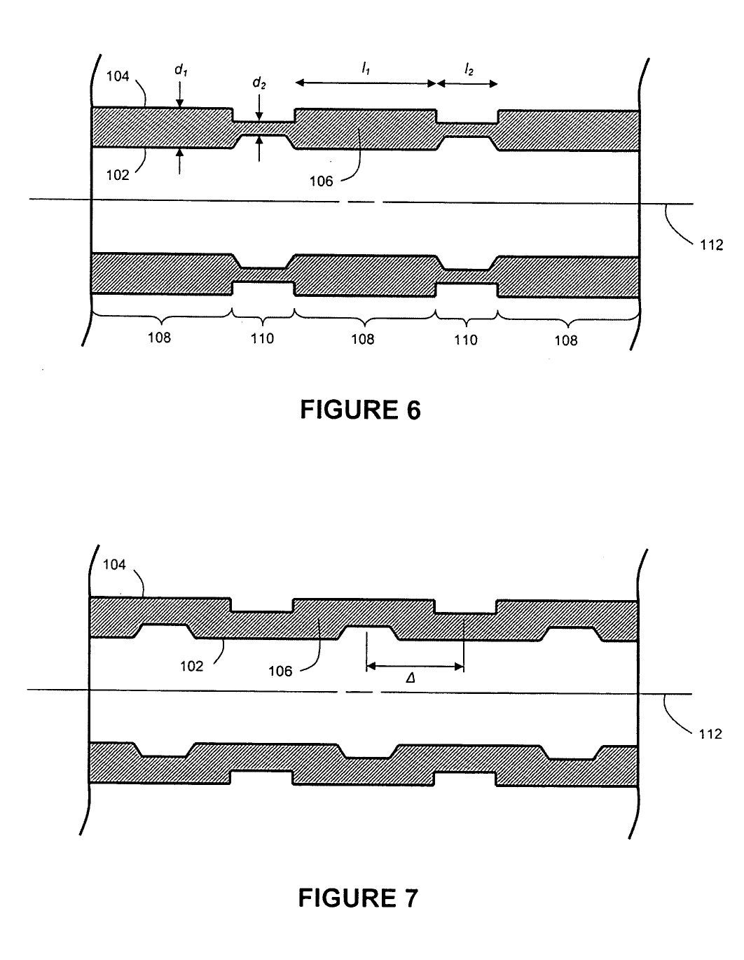

While embodiments have been described in which inner surface 102 is flat, in some embodiments, inner surface 102 can be non-flat (shaped). As an example, FIG. 4 shows an embodiment in which regions 110 are formed so that inner surface 102 is non-flat. As another example, as shown in FIG. 5, in certain embodiments, both surfaces 102 and 104 can be non-flat. In general, regions 110 can be formed so that surfaces 102 and/or 104 have features at the same or different locations along a direction of axis 112 with the same or different cross-sectional shapes. In some embodiments, for example, surfaces 102 and 104 have features with cross-sectional shapes that are all substantially similar (e.g., as shown in FIG. 5). As shown in FIG. 6, in some embodiments, surface 102 has features with cross-sectional shapes that are different from cross-sectional shapes of features of surface 104. In some embodiments, regions 110 can be formed so that surfaces 102 and 104 have features that are aligned with one another along a direction of axis 112 (e.g., FIG. 6). In certain embodiments, such as shown in FIG. 7, regions 110 can be formed so that surfaces 102 and 104 have features that are not all aligned with one another (offset by an amount A along a direction of axis 112) along a direction of axis 112.

Regions 108 and 110 of stent 100 can be prepared using any desired technique, such as, for example, mechanical machining, laser machining, electron beam etching, and/or chemical etching processes.

In some embodiments, mechanical forces such as external physiological stresses imparted to a stent by the lumen environment can be concentrated in second regions 110 of stent 100 by selecting a particular cross-sectional profile of second regions 110, increasing an erosion rate of second regions 110 relative to first regions 108. Mechanical forces can also arise from an internal structure of stent 100. For example, compressive and/or tensile stress can be introduced into a stent during manufacture, and the compressive and/or tensile stress can be used to control a rate of erosion of the stent. In general, regions of a stent that have residual compressive stress, e.g., regions that are compressed relative to a bulk structure of the stent material, have an erosion rate in a body lumen that is smaller than an erosion rate of an unstressed bulk material that has the same chemical composition. Further in general, regions of a stent that have residual tensile stress, e.g., regions that are stretched relative to a bulk structure of the stent material, have an erosion rate in a body lumen that is larger than an erosion rate of an unstressed bulk material that has the same chemical composition. By introducing compressive and/or tensile residual stress in a stent, the size and/or shape of stent degradation fragments resulting from erosion can be controlled.

Residual tensile stress can be introduced into a stent by straining the stent tubing as the final manufacturing operation. This can be done, for example, by pulling the tube through a die that causes a reduction in area of from 5% to 20%. Residual compressive stress can be introduced into a stent material by mechanical processing of the stent using techniques such as shot peening and/or grit blasting. These techniques can be applied to specific regions of a stent. In some embodiments, it may be easier to manufacture stents having residual compressive stress than stents having residual tensile stress, and therefore manufacturing techniques can be applied to produce regions 108 rather than regions 110. Regions 110 can, in certain embodiments, include unstressed stent material. For example, shot peening and/or grit blasting techniques can be used to produce regions 108 of stent 100, where regions 108 have a smaller erosion rate than regions 110 of stent 100. Residual tensile stress can be introduced by stretching portions of stent 100 during manufacture. Alternatively, or in addition, residual tensile stress can be introduced in regions of stent 100 by heating the stent and then subsequently cooling the stent by employing different cooling rates in different regions of stent 100 to impart different amounts of residual tensile stress in the different regions. Residual stress, e.g., residual compressive stress and/or residual tensile stress, can be introduced into regions of stent 100 adjacent to inner surface 102, adjacent to outer surface 104, and/or within a bulk region of stent wall 106.

The magnitude of residual stress that is created in stent 100 can be expressed as a percentage of the yield strength. The range of compressive residual stress is generally from 5% to 70% of the annealed material yield strength. It can be desirable for the compressive residual stress that would serve to decrease the degradation rate to be in the range of from 10% to 50% of the annealed material yield strength. The process for introducing the residual stress can be designed by a shot peening or blasting company such as Metal Improvement Company, Inc. (Teaneck, N.J.). The shot peening can be performed on flat strips of magnesium that can then be subsequently rolled into tubular shape and seam welded. The rolled and welded tubes can then be used for stent manufacturing. The shot peening of strips can allow both sides of the strip to be treated resulting in the OD and ID surfaces of the stent to have the residual stresses. Seamless magnesium tubes can be shot peened on the OD surface prior to stent manufacturing. The stents can then have OD surfaces with residual stresses and untreated ID surfaces. This can cause the stent to deteriorate from the ID towards the OD through the wall. Localized areas of shot peened surfaces can be annealed with a laser to relieve the residual stresses and make those specific areas degrade at a faster rate than adjacent areas where the residual stresses remained.

In some embodiments, stent 100 can have multiple different regions 108 having different amounts of residual compressive stress. Alternatively, or in addition, in some embodiments, stent 100 can have multiple different regions 110 having different amounts of residual tensile stress. The multiple different regions 108 and regions 110 can be positioned relative to one another in stent 100 to control a rate of erosion of stent 100 in a body lumen, and/or to control the morphologies of fragments of stent 100 that result from erosion.

In certain embodiments, differentiation in erosion rate in a body lumen can be achieved for a stent without the presence of structural features (e.g., without the presence of regions 108 and 110 having different thicknesses). In some embodiments, stent 100 can have regions with residual compressive and/or tensile stress, and can also have structural features such as notches and other surface relief features to provide additional control over an erosion rate of stent 100 and the morphologies of fragments of stent 100 resulting from erosion. For example, regions 110 can be formed so that they define notch-shaped recesses in a wall of stent 100. Further, regions 110 can include residual tensile stress introduced during manufacture of stent 100. The combination of notch-shaped recesses and residual tensile stress in second regions 110 can increase an erosion rate of second regions 110 relative to first regions 108, and can also decrease an erosion time of second regions 110 relative to first regions 108 in a body lumen.

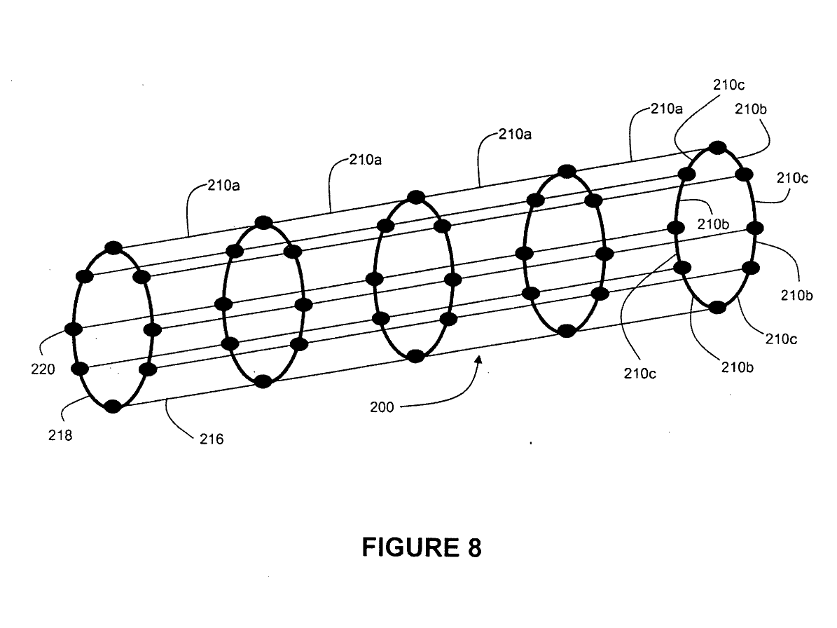

In some embodiments, it may be desirable to include more than two different types of regions having properties tailored to control fragmentation of a stent. A schematic diagram of a stent 200 is shown in FIG. 8. Stent 200 includes strut members 216 and ring members 218 joined at connection points 220 so that stent 200 has regions 210a, 210b and 210c having different erosion and/or fragment characteristics. This arrangement can allow stent 200 to fragment in a desired fashion.

For example, the three different types of regions 210a, 210b, and 210c can be selected to provide for different average erosion times in each of the different types of second regions, so that fragmentation of stent 200 within a body lumen can occur in different regions of stent 200 as a function of time. As an example, the properties of regions 210a can be selected to provide for the smallest average erosion time from among regions 210a, 210b, and 210c. As another example, the properties of regions 210b can be selected to provide for the next smallest average erosion time from among regions 210a, 210b, and 210c. As an additional example, the properties of regions 210c can be selected to provide for the largest average erosion time from among regions 210a, 210b, and 210c.

Different average erosion times for regions 210a, 210b, and 210c can be achieved in a variety of ways. For example, in some embodiments, regions 210a, 210b, and 210c have different thicknesses which lead to different average erosion times of these regions in a body lumen (e.g., regions 210a being thinnest and regions 210c being thickest). Alternatively, or in addition, in some embodiments, cross-sectional shapes of regions 210a, 210b, and 210c can vary in order to produce different average erosion times for the three types of regions. For example, regions 210a can have trapezoidal cross-sectional shapes, and regions 210b and 210c can have rectangular cross-sectional shapes. Geometrical dimensions of the cross-sectional shapes of each of regions 210a, 210b and 210c can also be selected as desired to produce different average erosion times for each of the three types of regions.

In some embodiments, successive regions 210b are displaced from one another along a ring circumference by angular increments (e.g., of 90 degrees). In certain embodiments, regions 210c are separated from adjacent regions 210b by angular increments (e.g., of 45 degrees). Linear spacings between adjacent regions 210a, and angular spacings between successive regions 210b and between adjacent regions 210b and regions 210c can, in general, be selected as desired to produce a particular fragmentation pattern when stent 200 undergoes erosion in a body lumen.

Fragments of stent 200 produced from erosion in regions 210a can include rings 218 having pieces of struts 216 attached via connectors 220, and, in some embodiments, smaller free pieces of struts 216 as well. If the average erosion time of secondary second regions 210b is shorter than the average erosion time of tertiary second regions 210c, erosion of regions 210b will typically occur next, resulting in arc-shaped fragments of stent material with pieces of strut material attached. As erosion of stent 200 continues further, additional stent fragments are produced from the arcs of rings 218 according to an angular increment between adjacent regions 210b and 210c.

### Compositional Features

While embodiments have been described in which the erosion of a stent in a body lumen is controlled via structural and/or mechanical features, in some embodiments, erosion of a stent can be controlled by selectively manipulating features relating to the composition of various regions of the stent. For example, an erosion rate of a stent can be controlled by varying a distribution of solid structural phases within regions of the stent.

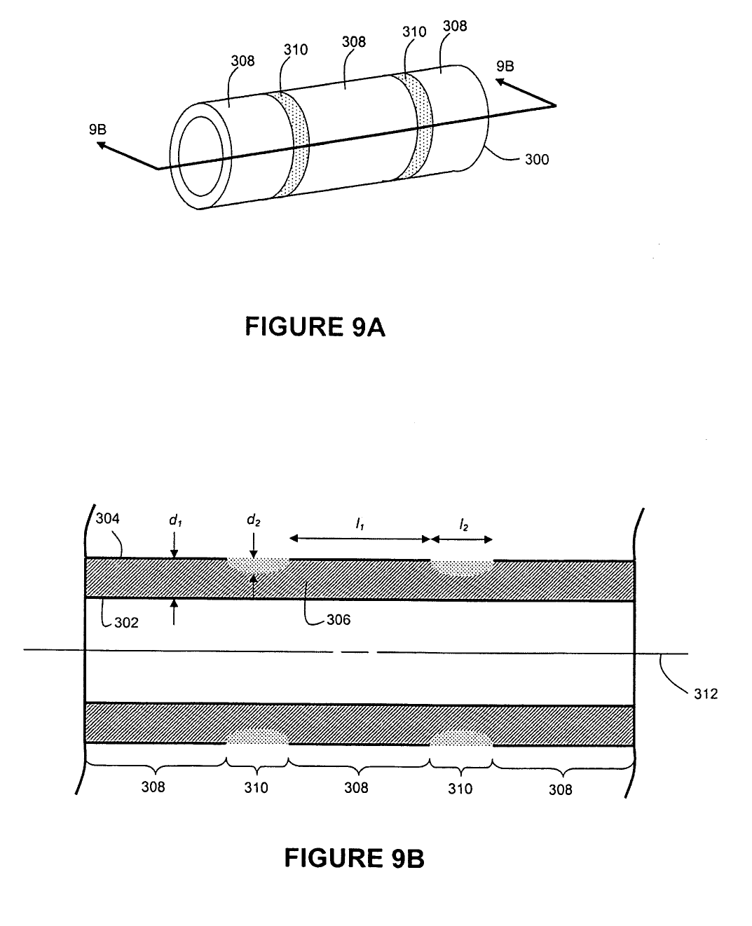

FIGS. 9A and 9B show perspective and cross-sectional views, respectively, of a stent 300. Stent 300 is tubular and has an inner surface 302 and an outer surface 304. The material between these surfaces forms a stent wall 306. In some embodiments, wall 306 is formed of one or more metal-containing materials, such as those discussed above.

In some embodiments, regions 308 of stent 300 are formed from one or more materials in a solid phase, and regions 310 are formed from the same material(s) as regions 308, but in a different solid phase than that of regions 308. In general, different solid phases of a stent material have different erosion rates in a body lumen.

In general, second regions 310 can be produced by processing selected regions of stent 300 to convert the stent material in the selected regions from a first solid phase to a second solid phase. For example, to produce regions 310 in a solid phase different from the solid phase of regions 308, regions 310 can be selectively heated using methods such as laser heating, electron beam heating, and electric arc heating. Heat sinks can be temporarily attached to portions (e.g., regions 308) to avoid changing the solid phase of regions 308 while regions 310 are heated to undergo a change in solid phase. Subsequently, rapid selective cooling of regions 310 can be used to prevent the material in regions 310 from reverting to the first solid phase. As a result, second regions 310 remain in the second phase, even when stent 300 returns to ambient temperature and pressure conditions.

As shown in FIG. 9B, regions 310 can be positioned adjacent outer surface 304 of stent 300. Alternatively, in some embodiments, regions 310 can be positioned adjacent to inner surface 302. In certain embodiments, regions 310 can be positioned adjacent to both inner and outer surfaces 302 and 304. Further, in some embodiments, second regions 310 adjacent to inner surface 302 can be aligned along a direction of longitudinal axis 312 of stent 300 with second regions 310 adjacent to outer surface 304. In other embodiments, second regions 310 adjacent to both inner surface 302 and outer surface 304 can be positioned so that second regions 310 adjacent to inner surface 302 are not aligned with corresponding second regions 310 adjacent to outer surface 304 along a direction of axis 312. For example, second regions 310 adjacent to inner surface 302 can be offset from second regions 310 adjacent to outer surface 304 by an average distance measured along a direction of axis 312.

In some embodiments, not all of the material in regions 310 is in a solid phase that is different from the solid phase of the material in regions 308. Further, portions of the material in a given solid phase in regions 310 may not be distributed uniformly among regions 310. Instead, portions of the material in a given solid phase can be distributed along grain boundaries or as precipitates within larger grains of stent material in a different solid phase (e.g., the solid phase of regions 308). In certain embodiments, portions of material in regions 310 that are in a different solid phase from regions 308 can be present in sufficiently larger concentrations so that they surround portions of the material in regions 310 that are in a different solid phase.

Without wishing to be bound by theory, differential erosion rates between regions 308 and 310 may be due to different structural morphologies between the regions as a result of various processing steps and/or techniques applied to selected regions of stent 300. For example, selected portions of stent 300 that include a metallic material having a wrought metal structure can be heated to transform the metal therein to a cast structural form. Typically, regions 310 that have cast metal structures erode at higher rates than regions 308 that have wrought metal structures, because cast metal structures generally feature coarser metallic grains, are less chemically homogeneous throughout, and may possibly feature multiple solid metal phases.

In some embodiments, regions 310 can include precipitates derived from one or more constituents of a material. For example, stent 300 can be formed from a material that includes an alloy of magnesium and zinc with small amounts of iron (e.g., from 0.1 weight percent iron to 5 weight percent iron). Heating selected portions of stent 300 to form regions 310 produces precipitates of pure iron in regions 310. Erosion mechanisms such as galvanic corrosion that occur within a body lumen can significantly reduce an erosion time of regions 310 relative to an erosion time of first regions 308. For example, magnesium and iron are widely separated on the galvanic series, so that when iron precipitates are formed in a magnesium matrix by selective heating of regions 310, galvanic corrosion between the magnesium and the iron precipitates can occur, leading to a higher erosion rate of regions 310 relative to regions 308.

In some embodiments, mixtures and/or solid solutions of different components of the stent material can be formed prior to forming the stent structure. In certain embodiments, additional materials such as iron can be added after stent 300 is formed from magnesium, from magnesium alloy, or from other materials. For example, materials such as iron can be sputtered onto portions of selected surfaces of stent 300, e.g., portions of inner surface 302 and/or portions of outer surface 304, and then diffused into the stent material using laser heating methods. This method can be used to produce accurately positioned, well-defined second regions 310 in stent 300.

In some embodiments, regions 310 have a thickness d₂ in a radial direction perpendicular to axis 312 of stent 300 that can be determined by a temperature depth profile during a heating process used to produce second regions 310. For example, d₂ can be 10 millimeters or less (e.g., 5 millimeters or less, 3 millimeters or less, 2 millimeters or less, 1.5 millimeters or less, 1 millimeter or less, 750 microns or less, 500 microns or less, 400 microns or less, 300 microns or less, 200 microns or less, 150 microns or less, 100 microns or less, 50 microns or less). Alternatively, or in addition, in certain embodiments, the thickness d₂ can be 10 microns or more (e.g., 20 microns or more, 50 microns or more, 100 microns or more, 150 microns or more, 200 microns or more, 300 microns or more, 400 microns or more, 500 microns or more, 750 microns or more, 1 millimeter or more, 1.5 millimeters or more, 2 millimeters or more, 3 millimeters or more, 5 millimeters or more).

In general, a length l₁ of first regions 308 in a direction of axis 312 can be selected as desired. The length of regions 308 can be selected, for example, to control a length of stent fragments resulting from erosion of stent 300 within a body lumen. Because erosion is typically complete in regions 310 before it is complete in regions 308, the length of regions 308 approximately determines the length of fragments of stent 300. For example, the length l₁ can be chosen to be 1 micron or more (e.g., 2 microns or more, 5 microns or more, 10 microns or more, 20 microns or more, 50 microns or more, 100 microns or more, 250 microns or more, 500 microns or more, 1 millimeter or more, 2 millimeters or more, 5 millimeters or more, 10 millimeters or more). Alternatively, or in addition, the length l₁ can be chosen to be 50 millimeters or less (e.g., 40 millimeters or less, 30 millimeters or less, 20 millimeters or less, 10 millimeters or less, 5 millimeters or less, 2 millimeters or less, 1 millimeter or less, 500 microns or less, 250 microns or less, 100 microns or less, 50 microns or less, 40 microns or less, 30 microns or less, 20 microns or less, 10 microns or less).

In general, a length l₂ of second regions 310 in a direction of axis 312 can be selected as desired to provide larger or smaller regions of stent 300 in which erosion of stent wall 306 is complete in a shorter time than erosion of stent wall 306 in regions 308. For example, length l₂ can be 10 millimeters or less (e.g., 8 millimeters or less, 6 millimeters or less, 4 millimeters or less, 2 millimeters or less, 1 millimeter or less, 750 microns or less, 500 microns or less, 250 microns or less, 150 microns or less, 100 microns or less, 50 microns or less, 20 microns or less, 10 microns or less, 5 microns or less, 2 microns or less, 1 micron or less). Alternatively, or in addition, length l₂ can be 1 micron or more (e.g., 5 microns or more, 10 microns or more, 20 microns or more, 50 microns or more, 100 microns or more, 150 microns or more, 250 microns or more, 500 microns or more 750 microns or more, 1 millimeter or more, 2 millimeters or more, 4 millimeters or more, 6 millimeters or more, 8 millimeters or more, 10 millimeters or more).

In certain embodiments, the properties of stent 300, including the chemical composition and material phases of first regions 308 and second regions 310 of stent 300, can be selected according to the type of the stent, to provide an average lifetime of stent 300 within a body lumen (e.g., an average time before erosion leads to failure of stent 300). For example, if stent 300 is a coronary stent, the stent material composition and phase in regions 310 can be chosen so that erosion of stent wall 306 in at least some of regions 310 is complete in a time from 3 months to 6 months following implantation of stent 300 into a coronary lumen. As another example, if stent 300 is a tracheal stent, the stent material composition and phase in regions 310 can be chosen so that erosion of stent wall 306 in at least some of regions 310 is complete in a time from 6 months to 24 months following implantation of stent 300 in a tracheal lumen.

In some embodiments, stent 300 can include multiple different types of regions 310 having different erosion rates. The multiple different types of regions 310 can correspond, for example, to stent material in multiple different solid phases. Erosion rates of each of the different types of regions 310 can be larger than an erosion rate of first regions 308. The multiple different types of regions 310 can be arranged, for example, on strut and ring members of a stent to create primary, secondary, and tertiary erosion regions (e.g., similar to as described in connection with FIG. 8). Erosion of stent 300 within a body lumen may then lead to initial formation of stent fragments that include ring members with portions of struts attached, followed subsequently by arc portions of the ring members, and then by smaller arc portions, as erosion continues.

In general, stent 300 can also have one or more of the features discussed in connection with stents 100 and 200. For example, second regions 310 can form notches or other surface features in one or more surfaces of stent 300 (e.g., inner surface 302 and/or outer surface 304). Regions 310 can further include different material phases or solid structures, and/or constituent precipitates. In some embodiments, regions 308 and/or regions 310 can include residual compressive or tensile stress. Combinations of features can be used to selectively prepare stents having desired sets of properties.

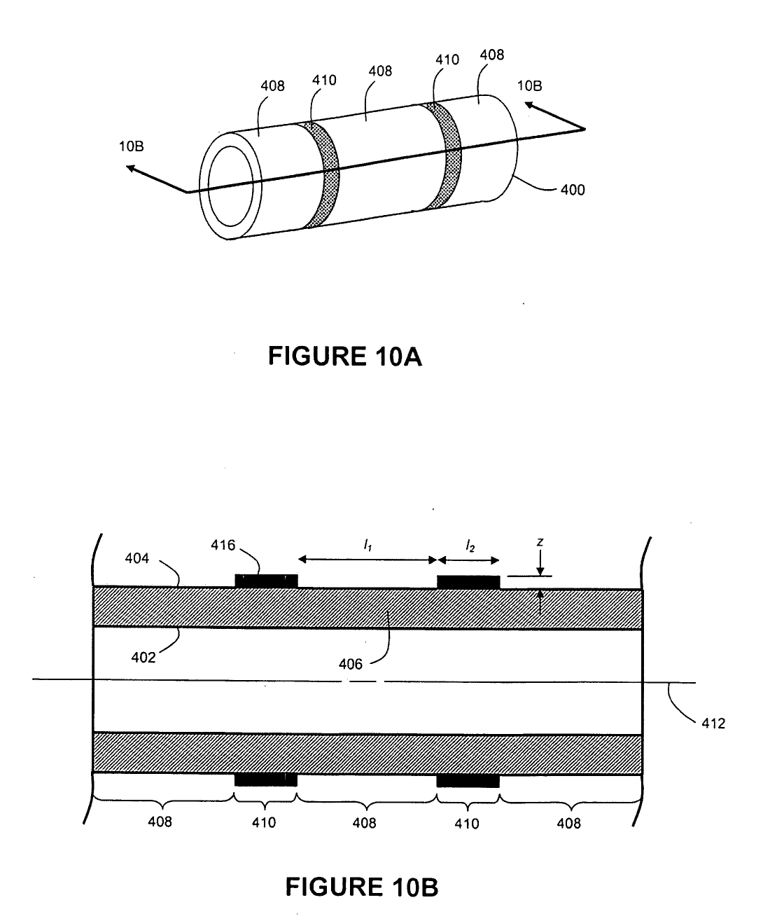

Erosion rates of stents can also be controlled by coating selected regions of one or more stent surfaces with additional materials to either reduce or increase an erosion rate of the coated regions within a body lumen. Perspective and cross-sectional views of a coated stent 400 are shown in FIGS. 10A and 10B, respectively. Stent 400 is tubular and has an inner surface 402 and an outer surface 404. The material between surfaces 402 and 404 forms stent wall 406.

Stent 400 includes first regions 408 that have a length l₁ in a direction of longitudinal axis 412 of stent 400, and second regions 410 that have a length l₂ in a direction of axis 412. One or more surfaces of second regions 410 are coated with a coating material 416.

In some embodiments, coating material 416 is selected to enhance an erosion rate of second regions 410 of stent 400, relative to an erosion rate of first regions 408. In general, coating material 416 is selected based on its chemical properties, and based on the material of stent 400. For example, if stent 400 includes magnesium, e.g., as magnesium metal or as a magnesium alloy, then coating material 416 can include at least one of iron or carbon steel. Each of these metals is separated from magnesium on the galvanic series, and galvanic corrosion can occur between coating material 416 and magnesium in the stent material in second regions 410. As a result of corrosion, an overall erosion rate of second regions 410 in a body lumen is larger than an erosion rate of first regions 408. Examples of metal-containing materials from which coating 416 can be formed include the metal-containing materials described above. In certain embodiments, coating material 416 can be a non-metallic material. For example, coating material 416 can be an organic material, such as an organic acid, an organic salt, an organic halide (e.g., an organic chlorine), an organic sodium material, or an organic potassium material.

Coating material 416 can generally be disposed on either inner surface 402 or outer surface 404 of stent 400, or on both inner and outer surfaces 402 and 404. For example, in FIG. 10B, coating material 416 is disposed on outer surface 404 of stent 400. In some embodiments, coated regions of inner surface 402 and outer surface 404 can be aligned with one another along a direction of longitudinal axis 412 of stent 400. In other embodiments, coated regions of inner surface 402 can be offset by an average distance measured along a direction of axis 412 from coated regions of outer surface 404.

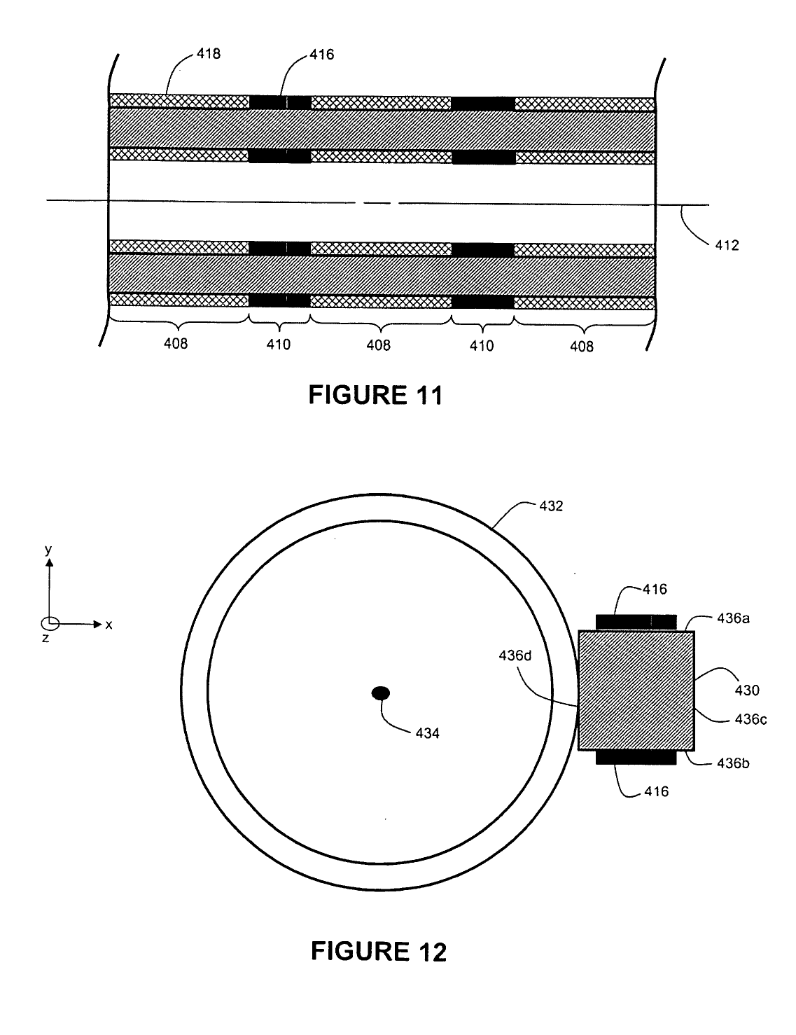

In certain embodiments, coating materials can be used to reduce an erosion rate of selected regions of stent 400, relative to uncoated stent regions. For example, in the embodiment 10 shown in FIG. 11, first regions 408 are coated with coating material 418 disposed on inner surface 402 and outer surface 404 of stent 400. Coating material 418 can be a material such as MgO₂ or MgF₂. For a magnesium stent, selected portions of the stent surface can be made to be less corrosion resistant than others so as to have increased deterioration rate there leading to disintegration at these preferred sites. Magnesium can be most highly corrosion resistant after chemical treatment in a ferric nitrate solution. Locations where increased deterioration rate is desired could be masked with a polymer sealant that is resistant to penetration by the ferric nitrate solution and then the entire part could be treated in the ferric nitrate solution. Upon removal of the maskant, locations would have been created that did not have the surface treatment and would then deteriorate more quickly than neighboring surface regions. Another method of achieving this outcome instead of using ferric nitrate solution would be to treat the part with masked areas in a chromate conversion coating or apply an electroplate deposit of a metal that is more corrosion resistant but still is biodegradable, such as iron or carbon steel.

Coating material 418 provides a barrier between the stent material, e.g., stent wall 406, and the surrounding environment of a body lumen. Due to the barrier provided by coating material 418, an erosion rate of first regions 408 is reduced relative to an erosion rate of uncoated regions of stent 400. A method of slowing the deterioration rate of a magnesium stent is to apply an anodization treatment to produce a surface oxide layer that contains microporosity. This can limit the interaction of the magnesium with the body fluids thereby having the stent maintain mechanical strength for a longer period of time (e.g., for an airway stent). Different degradation rates could be made within the stent by sealing the porosity in some areas of the anodized stent with a bioabsorbable polymer and leaving areas where higher degradation rate is desired unsealed.

In certain embodiments, both coating material 416 and coating material 418 can be used to coat selected portions of certain surfaces of stent 400. For example, in the embodiment shown in FIG. 11, inner and outer surfaces 402 and 404 of first regions 408 are coated with coating material 418, and inner and outer surfaces 402 and 404 of second regions 410 are coated with coating material 416.

In some embodiments, either or both of coating materials 416 and 418 can be disposed on inner and outer surfaces 402 and 404 of stent 400 in a patterned array. For example, coating material 416 can be disposed on surface 402 and/or surface 404 as a series of lines. The lines can extend in a single direction, e.g., parallel to axis 412, or along a circumference of stent 400. Alternatively, or in addition, coating material 416 can be deposited on surface 402 and/or surface 404 of stent 400 in a pattern that, when projected in two dimensions, is a regular pattern such as a rectangular grid pattern, for example, or a diamond-shaped pattern, or a hexagonal pattern, or a pattern having another desired configuration.

Coating materials 416 and 418 can be deposited on selected regions of surfaces of stent 400 using various deposition techniques. For example, coating materials 416 and 418 can be deposited using chemical vapor deposition, physical vapor deposition, or sputtering. In some embodiments, coating material 418 can be deposited by chemically reacting the stent material. For example, where stent 400 includes magnesium, a coating material 418 that includes magnesium fluoride can be deposited by exposing uncoated regions of stent 400 to a fluorine source. As another example, a coating material 418 that includes magnesium oxide can be deposited by heated selected uncoated regions of stent 400 and exposing the selected regions to oxygen.

The thickness t of coating materials 416 and 418 in a radial direction transverse to a direction of axis 412 can generally be selected as desired to control erosion rates of first regions 408 and second regions 410 of stent 400. For example, t can be 10 nm or more (e.g., 20 nm or more, 50 nm or more, 100 nm or more, 250 nm or more, 500 nm or more, 1 micron or more, 10 microns or more, 50 microns or more, 100 microns or more, 250 microns or more, 500 microns or more, 1 millimeter or more). In certain embodiments, the thicknesses of coating materials 416 and 418 are the same. In other embodiments, coating materials 416 and 418 can have different thicknesses, each of which can generally be selected as desired.

In general, a length l₁ of first regions 408 in a direction of axis 412 can be selected as desired. The length of regions 408 can be selected, for example, to control a length of stent fragments resulting from erosion of stent 400 within a body lumen. Because erosion is typically complete in regions 410 before it is complete in regions 408, the length of regions 408 approximately determines the length of fragments of stent 400. For example, the length l₁ can be chosen to be 1 micron or more (e.g., 2 microns or more, 5 microns or more, 10 microns or more, 20 microns or more, 50 microns or more, 100 microns or more, 250 microns or more, 500 microns or more, 1 millimeter or more, 2 millimeters or more, 5 millimeters or more, 10 millimeters or more). Alternatively, or in addition, the length l₁ can be chosen to be 50 millimeters or less (e.g., 40 millimeters or less, 30 millimeters or less, 20 millimeters or less, 10 millimeters or less, 5 millimeters or less, 2 millimeters or less, 1 millimeter or less, 500 microns or less, 250 microns or less, 100 microns or less, 50 microns or less, 40 microns or less, 30 microns or less, 20 microns or less, 10 microns or less).

In general, a length 12 of second regions 410 in a direction of axis 412 can be selected as desired to provide larger or smaller regions of stent 400 in which erosion of stent wall 406 is complete in a shorter time than erosion of stent wall 406 in regions 408. For example, length l₂ can be 10 millimeters or less (e.g., 8 millimeters or less, 6 millimeters or less, 4 millimeters or less, 2 millimeters or less, 1 millimeter or less, 750 microns or less, 500 microns or less, 250 microns or less, 150 microns or less, 100 microns or less, 50 microns or less, 20 microns or less, 10 microns or less, 5 microns or less, 2 microns or less, 1 micron or less). Alternatively, or in addition, length l₂ can be 1 micron or more (e.g., 5 microns or more, 10 microns or more, 20 microns or more, 50 microns or more, 100 microns or more, 150 microns or more, 250 microns or more, 500 microns or more 750 microns or more, 1 millimeter or more, 2 millimeters or more, 4 millimeters or more, 6 millimeters or more, 8 millimeters or more, 10 millimeters or more).

In certain embodiments, the properties of stent 400, including the coating materials 416 and/or 418 and their thicknesses, can be selected according to the type of the stent, to provide an average lifetime of stent 400 within a body lumen (e.g., an average time before erosion leads to failure of stent 400). For example, if stent 400 is a coronary stent, the coating materials 416 and 418 can be chosen so that erosion of stent wall 406 in at least some of second regions 410 is complete in a time from 3 months to 6 months following implantation of stent 400 into a coronary lumen. As another example, if stent 400 is a tracheal stent, the stent material composition and phase in regions 410 can be chosen so that erosion of stent wall 406 in at least some of regions 410 is complete in a time from 6 months to 24 months following implantation of stent 400 in a tracheal lumen.

In some embodiments, stent 400 can include multiple different types of second regions 410 having different erosion rates. The multiple different types of second regions 410 can correspond, for example, to different types of coating materials 416 and/or different thicknesses of coating materials 416. Erosion rates of each of the different types of regions 410 can be larger than an erosion rate of first regions 408. The multiple different types of regions 410 can be arranged, for example, on strut and ring members of a stent to create primary, secondary, and tertiary erosion regions, such as described in connection with FIG. 8. Erosion of stent 400 within a body lumen may then lead to initial formation of stent fragments that include ring members with portions of struts attached, followed subsequently by arc portions of the ring members, and then by smaller arc portions, as erosion continues.

In some embodiments, coating materials can be disposed on selected regions of stent surfaces to control an erosion cross-section of stent 400 or portions thereof. For example, one or more coating materials can be deposited on selected surfaces of stent members in order to impart direction-specific mechanical properties to the members as erosion occurs within a body lumen. FIG. 12 shows a cross-sectional view of a strut member 430. Strut member 430 has a substantially rectangular cross-sectional shape. Ring member 432 is also shown, but is not in the plane of FIG. 12. Longitudinal axis 434 is oriented perpendicular to the plane of FIG. 12. Coating material 416 is deposited on surfaces 436a and 436b of strut member 430. An erosion rate of strut member 430 in a direction parallel to they axis is larger than an erosion rate of strut member 430 in a direction parallel to the x axis due to coating material 416. As a result, erosion over a period of time of strut member 430 within a body lumen leads to strut member 430 assuming a cross-sectional shape that corresponds roughly to an “I-beam” shape. The resulting I-beam shaped strut member 430 retains mechanical strength and resists fracturing under physiological stress for a longer time in a radial direction, e.g., in the x-y plane, than in an axial direction (e.g., along the z axis).

In some embodiments, four surfaces of strut member 430 can be coated with coating material 416. For example, in certain regions of strut member 430, opposite surfaces 436a and 436b can be coated with coating material 416. In adjacent regions of strut member 430, opposite surfaces 436c and 436d can be coated with coating material 416. Erosion within a body lumen produces a strut member 430 that has a cross-sectional profile that varies in two different directions in the x-y plane. Asymmetric physiological loading of such strut members 430 can facilitate controlled fragmentation of stent 400 due to a multiplicity of regions of reduced mechanical strength along axis 434.

In certain embodiments, intersecting surfaces of stent members can be coated with coating material 416. For example, if the intersecting surfaces of strut member 430, e.g., the corners of strut member 430 in the cross-sectional view shown in FIG. 12, are coated with coating material 416, erosion of strut member 430 within a body lumen leads to strut member 430 assuming a diamond-shaped cross-sectional profile.

In some embodiments, selected surfaces of stent members can be coated with coating material 418 to reduce an erosion rate of the coated stent members along particular directions. In general, by controlling deposition of coating materials 416 and 418 on specific surfaces, such as strut and ring members, direction-dependent mechanical properties can be imparted to stents undergoing erosion and mechanical failure of the stent can be selected to occur along preferred directions in certain regions of the stent.

In general, stent 400 can also have one or more of the features discussed in connection with stents 100, 200, and/or 300. For example, regions 410 can form notches or other surface features in inner surface 402 and/or outer surface 404 of stent 400. Regions 410 can further be coated with coating material 416 to increase an erosion rate of regions 410 relative to uncoated regions of stent 400. First regions 408 and regions 410 can further include residual compressive and/or tensile stress. Regions 408 and 410 can include stent materials in different solid phases or having different structural morphologies. Combinations of features can be used to selectively manufacture stents having desired properties.

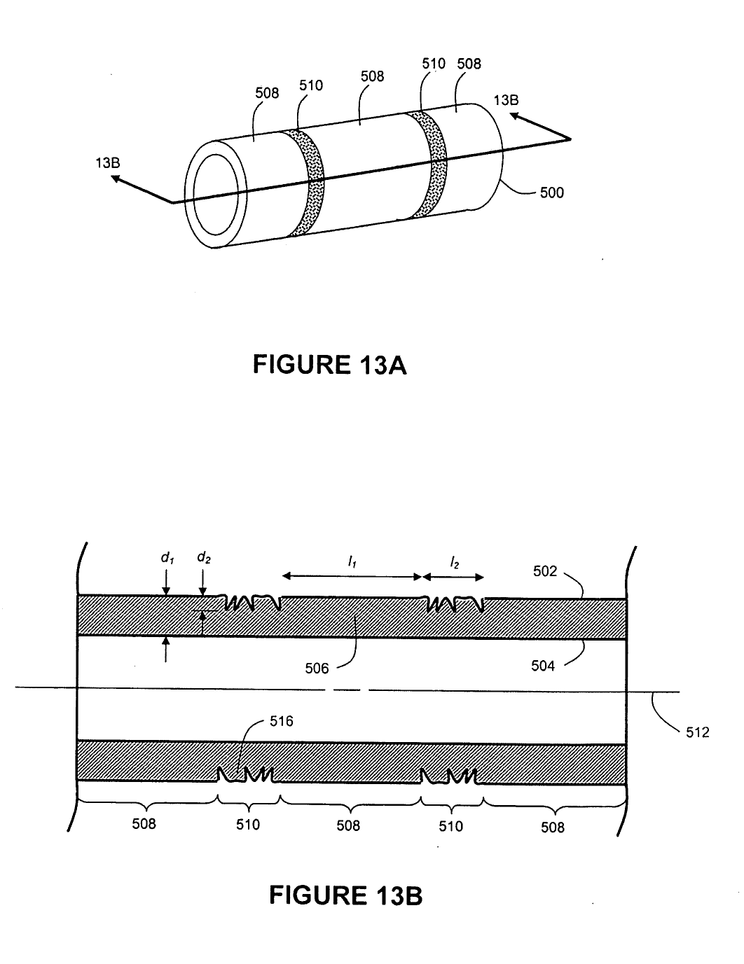

In some embodiments, the rate of erosion of a stent in a body lumen can be controlled by introducing pores in selected regions of the stent. Perspective and cross-sectional views of an embodiment of a stent 500 that includes pores are shown in FIGS. 13A and 13B, respectively. Stent 500 is tubular and includes an inner surface 502 and an outer surface 504. A stent wall 506 is formed by the stent material between surfaces 502 and 504. A plurality of pores 516 are located at outer surface 504 in regions 510 of stent 500. First regions 508 of stent 500 do not include pores.

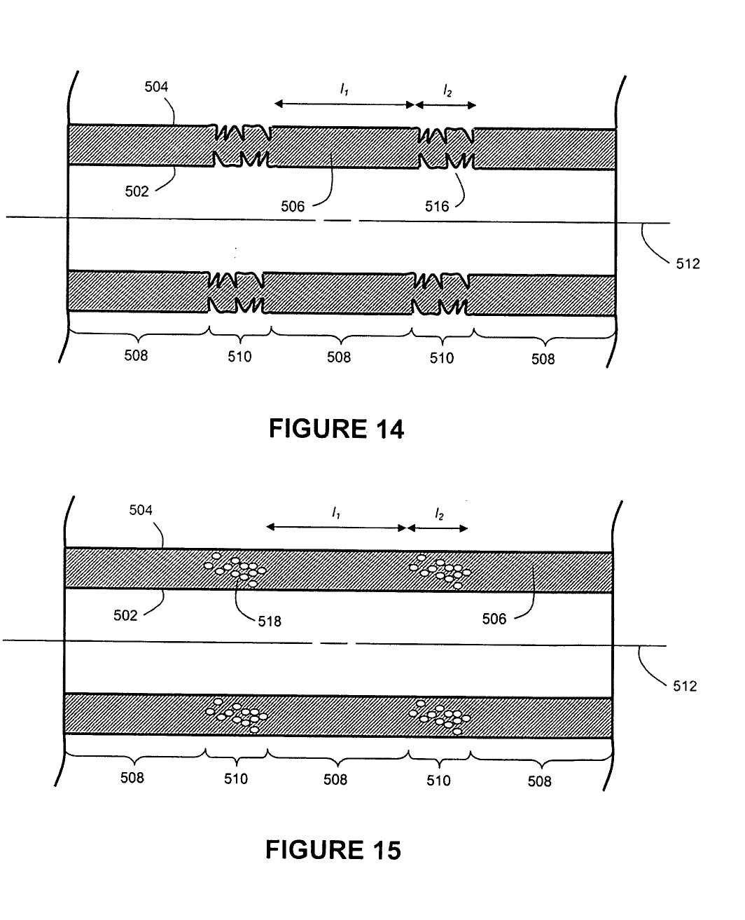

In some embodiments, pores can be located on inner surface 502 of stent 500 in alternative to, or in addition to, pores located on outer surface 504. For example, FIG. 14 is a cross-sectional view of an embodiment of stent 500 that includes pores 516 on both inner surface 502 and outer surface 504. In certain embodiments, regions of stent 500 that include pores on outer surface 504 can be aligned along a direction of longitudinal axis 512 with regions of stent 500 that include pores on inner surface 502. In other embodiments, regions of stent 500 that include pores on outer surface 504 are offset from regions of stent 500 that include pores on inner surface 502 by an average amount measured in a direction of axis 512. In general, the number of regions that include pores adjacent to outer surface 504 and the number of regions that include pores adjacent to inner surface 502 can be the same, or different.

In some embodiments, in addition to or in alternative to pores on one or more surfaces of stent 500, pores can be disposed entirely within stent wall 506. A cross-sectional view of an embodiment of stent 500 that has pores 518 entirely within stent wall 506 is shown in FIG. 15. In certain embodiments, both pores 518 and pores 516 can be present in stent 500.

In general, regions 510 of stent 500 erode at a faster rate within a body lumen than first regions 508. Without wishing to be bound by theory, a possible explanation for this effect is that pores increase the effective surface area of regions of stent 500 that contain them. Many processes that contribute to erosion of stent 500 in a body lumen occur at surfaces, and so regions of stent 500 that have a relatively larger surface area will erode faster than regions that have a relatively smaller surface area. Erosion rates of porous regions of stent 500 can be controlled by controlling distributions of pore diameters, and by controlling pore densities per unit volume, in the regions of stent 500 that have pores.

When stent 500 is inserted into a body lumen, erosion of stent 500 leads to formation of a plurality of stent fragments. The distribution of pore diameters and pore density in regions 510 can be selected so that erosion of stent 500 is complete in at least some of regions 510 before it is complete in regions 508. In general, stent 500 can include as many regions 508 and regions 510 as desired. Stent 500 can be formed from a variety of different materials, such as those discussed above.

Various methods can be used to introduce pores in selected regions of stent 500. One such method uses galvanic corrosion to introduce surface pores in selected regions of a metal stent 500. Selected regions of stent 500 are first protected by depositing a mask coating over exposed surfaces of the selected regions, e.g., portions of surfaces 502 and 504, for example. Stent 500 is then placed in an electrolyte solution, e.g., a saline solution with 9-27 g/l NaCl in de-ionized water, and an electric potential of 0.1 to 1.2V is applied to the solution to cause an electric current to flow for a time period of 5 seconds to 15 minutes, and to initiate galvanic corrosion of the regions of stent 500 where surfaces 502 and/or 504 are unprotected. The galvanic corrosion process introduces pores in the unprotected surfaces of stent 500. An average depth, diameter, and density of the pores can be controlled by adjusting the applied voltage, the temporal duration of the corrosion process, and the composition of the saline solution. In a final step, stent 500 is removed from the saline solution and the mask coating is removed. The resulting stent includes first regions 508 having nominally uniform, uncorroded inner and outer surfaces 502 and 504, and second regions 510 having a plurality of pores disposed on inner and/or outer surfaces 502 and 504 of stent 500.

Another method for introducing pores in selected regions of stent 500 includes heating stent 500 to a temperature higher than a melting temperature of the stent material, bubbling a gas through the melted stent material, and then cooling the heated stent material under conditions sufficient to trap gas (e.g., as bubbles) in the heated regions of the material. Gases used for this process can include noble gases such as argon, helium, and neon, and other gases such as nitrogen. Pores introduced using this method can include both pores that lie entirely within stent wall 506, and pores at inner and/or outer surfaces 502 and 504 of stent 500. In general, the pore density and average pore diameter in the selected regions of stent 500 can be controlled by adjusting a flow rate of the gas, and by selecting appropriate geometric properties of a bubbling system used to produce the gas bubbles.

An additional method for introducing pores in selected regions of stent 500 is leaching out one or more constituents from portions of stent 500 using methods such as filiform corrosion, and powder sintering. A further method includes introducing microbeads in melted regions of stent 500, and subsequently removing the microbeads when the melted regions have been cooled and have re-solidified.

In some embodiments, pores in second regions 510 can have a mean pore diameter of at least 10 nanometers (e.g., at least 20 nm, at least 50 nm, at least 100 nm), and/or at most 30 microns (e.g., at most 20 microns, at most 10 microns, at most 1 micron).

In certain embodiments, a density of pores per unit volume in second regions 510 can be at least 5% (e.g., at least 10%, at least 15%, at least 20%), and/or at most 60% (e.g., at most 50%, at most 40%, at most 30%).

In general, a length l₁ of regions 508 in a direction of axis 512 can be selected as desired. The length of regions 508 can be selected, for example, to control a length of stent fragments resulting from erosion of stent 500 within a body lumen. Because erosion is typically complete in regions 510 before it is complete in regions 508, the length of regions 508 approximately determines the length of fragments of stent 500. For example, the length l₁ can be chosen to be 1 micron or more (e.g., 2 microns or more, 5 microns or more, 10 microns or more, 20 microns or more, 50 microns or more, 100 microns or more, 250 microns or more, 500 microns or more, 1 millimeter or more, 2 millimeters or more, 5 millimeters or more, 10 millimeters or more). Alternatively, or in addition, the length l₁ can be chosen to be 50 millimeters or less (e.g., 40 millimeters or less, 30 millimeters or less, 20 millimeters or less, 10 millimeters or less, 5 millimeters or less, 2 millimeters or less, 1 millimeter or less, 500 microns or less, 250 microns or less, 100 microns or less, 50 microns or less, 40 microns or less, 30 microns or less, 20 microns or less, 10 microns or less).

In general, a length l₂ of regions 510 in a direction of axis 512 can be selected as desired to provide larger or smaller regions of stent 500 in which erosion of stent wall 506 is complete in a shorter time than erosion of stent wall 506 in regions 508. For example, length l₂ can be 10 millimeters or less (e.g., 8 millimeters or less, 6 millimeters or less, 4 millimeters or less, 2 millimeters or less, 1 millimeter or less, 750 microns or less, 500 microns or less, 250 microns or less, 150 microns or less, 100 microns or less, 50 microns or less, 20 microns or less, 10 microns or less, 5 microns or less, 2 microns or less, 1 micron or less). Alternatively, or in addition, length l₂ can be 1 micron or more (e.g., 5 microns or more, 10 microns or more, 20 microns or more, 50 microns or more, 100 microns or more, 150 microns or more, 250 microns or more, 500 microns or more 750 microns or more, 1 millimeter or more, 2 millimeters or more, 4 millimeters or more, 6 millimeters or more, 8 millimeters or more, 10 millimeters or more).

In some embodiments, stent 500 can have pores in both regions 508 and second regions 510. For example, pores can selectively be introduced into regions 508 using methods such as the methods disclosed above. Subsequently, pores can selectively be introduced into second regions 510 in such a manner that pores in regions 508 are unchanged. The properties of pores in each of regions 508 and regions 510 can be selected independently. For example, pores in first regions 508 can have a smaller mean diameter than pores in second regions 510. As another example, regions 508 can include fewer pores per unit volume than second regions 510.

In certain embodiments, the properties of stent 500, including the properties of pores in selected regions of stent 500, can be selected according to the type of the stent, to provide an average lifetime of stent 500 within a body lumen (e.g., an average time before erosion leads to failure of stent 500). For example, if stent 500 is a coronary stent, properties of pores in regions 510 can be chosen (e.g., by introducing a selected pore density per unit volume and/or a selected mean pore diameter in regions 510) so that erosion of stent wall 506 is complete in at least some of regions 510 in a time from 3 months to 6 months following implantation of stent 500 into a coronary lumen. As another example, if stent 500 is a tracheal stent, properties of pores in regions 510 can be chosen (e.g., by introducing a selected pore density per unit volume and/or a selected mean pore diameter) so that erosion of stent wall 506 is complete in at least some of regions 510 is complete in a time from 6 months to 24 months following implantation of stent 500 in a tracheal lumen.

In some embodiments, stent 500 can include multiple different types of regions 510 having different erosion rates. The multiple different types of regions 510 can correspond, for example, to different mean pore diameters and/or different pore densities. Erosion rates of each of the different types of regions 510 can be larger than an erosion rate of regions 508. The multiple different types of regions 510 can be arranged, for example, on strut and ring members of a stent to create primary, secondary, and tertiary erosion regions, as described in connection with FIG. 8. Erosion of stent 500 within a body lumen may then lead to initial formation of stent fragments that include ring members with portions of struts attached, followed subsequently by arc portions of the ring members, and then by smaller arc portions, as erosion continues. Pores can also be selectively introduced on individual stent member surfaces so that erosion of stent 500 within a body lumen changes a cross-sectional profile of selected stent members over time.

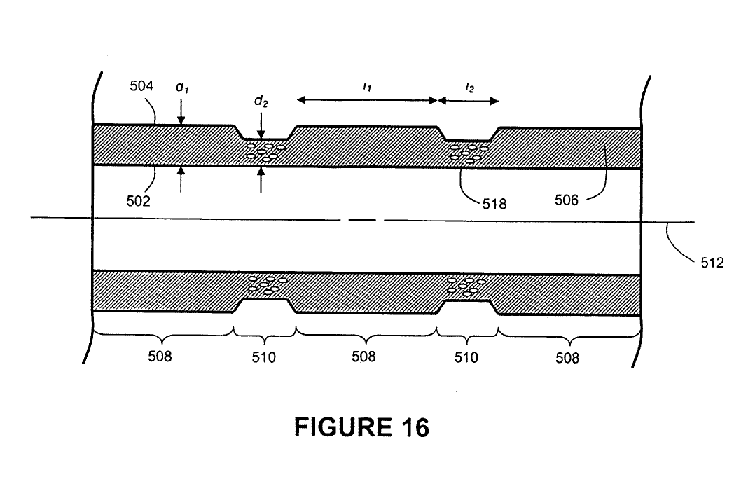

In general, stent 500 can have one or more of the features discussed in connection with stents 100, 200, 300, and 400. An embodiment of stent 500 that has a combination of surface features and regions with pores is shown in FIG. 16. Regions 508 and 510 of stent 500 include a stent material, and second regions 510 further include pores that lie entirely within stent wall 506 and/or pores in surfaces 502 and/or 504. In addition, a thickness d₂ of second regions 510 in a radial direction transverse to longitudinal axis 512 of stent 500 is less than a thickness d₁ of first regions 508. In certain embodiments, thickness d₂ can be 25% or more (e.g., 30% or more, 40% or more, 50% or more, 60% or more, 75% or more, 90% or more, 95% or more) of thickness d₁. Other geometrical and compositional parameters of stent 500 can be similar to those already discussed, for example.

In general, embodiments of stent 500 can include structural variations (e.g., surface features), residual compressive and tensile stress, multiple material phases and/or structural morphologies, coated regions, and porous regions, and these various structural and compositional features can be combined to control erosion of stent 500 in a body lumen. The disclosed features can be used in combination to manufacture stents that have desired properties.

### Stent Delivery Systems

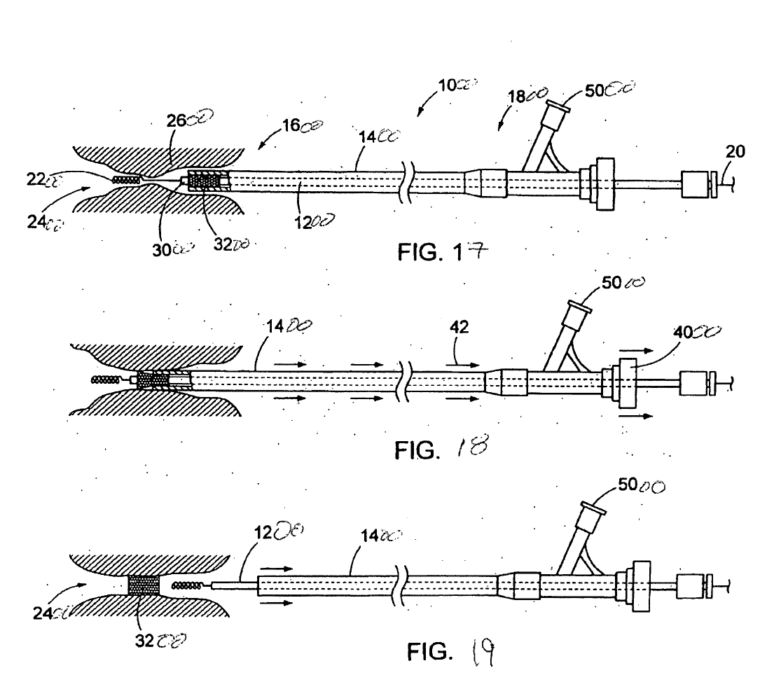

As noted above, the stents described herein can be, for example, self-expanding stents. FIGS. 17-19 show a system 1000 designed to deliver a self-expanding stent 3200 into a body lumen 2400 (e.g., an artery of a human). System 1000 includes a catheter 1200, a sheath 1400 surrounding catheter 1200. Stent 3200 is positioned between catheter 1200 and sheath 1400. System 1000 includes a distal end 1600 dimensioned for insertion into body lumen 2400 and a proximal end 1800 that resides outside the body of a subject. Proximal end 1800 has at least one port 5000 and lumens for manipulation by a physician. A guide wire 2000 with a blunted end 2200 is inserted into body lumen 2400 by, for example, making an incision in the femoral artery, and directing guide wire 2000 to a constricted site 2600 of lumen 2400 (e.g., an artery constricted with plaque) using, for example, fluoroscopy as a position aid. After guide wire 2000 has reached constricted site 2600 of body lumen 2400, catheter 1200, stent 3200 and sheath 1400 are placed over the proximal end of guide wire 2000. Catheter 1200, stent 3200 and sheath 1400 are moved distally over guide wire 2000 and positioned within lumen 2400 so that stent 3200 is adjacent constricted site 2600 of lumen 2400. Sheath 1400 is moved proximally, allowing stent 3200 to expand and engage constricted site 2600. Sheath 1400, catheter 1200 and guide wire 2000 are removed from body lumen 2400, leaving stent 3200 engaged with constricted site 2600.

### EXAMPLE

### Example 1

A magnesium tube is manufactured by conventional extrusion, pilgering, mandrel drawing, plug drawing or by rolling, seam-welding, and mandrel or plug drawing. The finished tube size is 0.090″ OD with a wall thickness of 0.0060″. The finished tube is in the annealed condition. The stent strut pattern is laser cut into the tubing. A focused Nd-YAG laser is used to scribe grooves into the OD surface of the laser cut stent. The grooves are positioned at locations of the stent where disintegration is desired to occur first. In this example, the grooves are positioned on the OD surface of every connector between strut rings. The grooves are made to a depth of from 20% to 30% of the wall thickness (from 0.0012 inch to 0.0018 inch). The laser-cut grooves are from one micron to 10 microns wide. Post-laser metal removal is performed by etching and electropolishing to remove the laser-affected material and to produce a smooth surface finish. Upon implantation, the physiological environment causes metal deterioration. Grooved locations thin down first to a thickness where the applied stresses exceeds the load-bearing capability of the material thickness and fracture occurs. The stent is thereby broken into individual rings which subsequently degrade and disintegrate into small fragments and are eventually harmlessly bioabsorbed.

### Example 2

A 0.0032″ thick annealed 1010 steel strip is shot peened on both sides to an Almen Intensity of from 0.010 inch to 0.014 inch A. The shot peened strip is rolled into a tubular shape and seam-welded. The welded tube is stress relieved at 400° F. The finished tube size is 0.072 inch outer diameter. The stent strut pattern is laser cut into the tubing. A focused Nd-YAG laser is used to locally anneal the tubing OD surface of the stent wherever initial fragmentation upon degradation is desired. In this example, the annealed spots are positioned on the OD surface of every connector between strut rings. Post-laser metal removal is performed by etching and electropolishing to remove the laser-affected material and to produce a smooth surface finish. The depth of the shot peened residual stress layer exceeds the post-laser metal removal envelope. Upon implantation, the physiological environment causes metal deterioration. Metal degradation occurs at the anneal spots at a faster rate than on the peened surfaces. Fracture occurs first at laser annealed locations when the applied stresses exceeds the load-bearing capability of the thinned material. The stent is thereby broken into individual rings which subsequently degrade and disintegrate into small fragments and are eventually harmlessly bioabsorbed.

### Example 3

A magnesium tube is manufactured by conventional extrusion, pilgering, mandrel drawing, plug drawing or by rolling, seam-welding, and mandrel or plug drawing. The finished tube size is 0.090″ OD with a wall thickness of 0.0060 inch. The finished tube is in the annealed condition. The stent strut pattern is laser cut into the tubing. A focused Nd-YAG or Excimer laser is used to superficially melt targeted portions of the OD surface of the laser cut stent. The melted areas are positioned at locations of the stent where disintegration is desired to occur first. In this example, the melted areas are positioned on the OD surface of every connector between strut rings. The melted spots are made to a depth of from 20% to 30% of the wall thickness (0.0012 inch to 0.0018 inch). The melted spots or bands are from one micron to 10 microns wide. Post-laser metal removal is performed by etching and electropolishing to remove the laser-affected material, except for portions of the melted spots, and to produce a smooth surface finish. Upon implantation, the physiological environment causes metal deterioration. Melted spots and bands thin down first to a thickness where the applied stresses exceeds the load-bearing capability of the material thickness and fracture occurs. The stent is thereby broken into individual rings which subsequently degrade and disintegrate into small fragments and are eventually harmlessly bioabsorbed.

### Example 4

A magnesium tube is manufactured by conventional extrusion, pilgering, mandrel drawing, plug drawing or by rolling, seam-welding, and mandrel or plug drawing. The finished tube size is 0.090 inch outer diameter with a wall thickness of 0.0060 inch. The finished tube is in the annealed condition. The stent strut pattern is laser cut into the tubing. Post-laser metal removal is performed by etching and electropolishing to remove the laser-affected material and produce a smooth surface finish. Areas of the stent that are desired to degrade more rapidly, such as connector struts, are masked with vinyl or polyethylene. The finished stent is then immersed for from one minute to three minutes in a ferric nitrate solution (180 g/L CrO₃, 40 g/L Fe(NO₃)-9H₂O, 3.5 g/L NaF, 16-38° C.). (Metals Handbook, Ninth Edition, Volume 5 Surface Cleaning, Finishing, and Coating, American Society for Metals, 1982, p.630.) The maskant is peeled off. Upon implantation, the physiological environment causes metal deterioration. Locations that had been masked thin down first to a thickness where the applied stresses exceeds the load-bearing capability of the material thickness and fracture occurs. The stent is thereby broken into individual rings which subsequently degrade and disintegrate into small fragments and are eventually harmlessly bioabsorbed.

### Example 5

A magnesium tube is manufactured by conventional extrusion, pilgering, mandrel drawing, plug drawing or by rolling, seam-welding, and mandrel or plug drawing. The finished tube size is 0.090 inch outer diameter with a wall thickness of 0.0060 inch. The finished tube is in the annealed condition. The stent strut pattern is laser cut into the tubing. Post-laser metal removal is performed by etching and electropolishing to remove the laser-affected material and to produce a smooth surface finish. A focused liquid spray nozzle is used to paint lines into the outer diameter surface of the laser cut stent. The material applied is a mixture of bioabsorbable polymer and fine NaCl or KCl crystals. The lines are positioned at locations of the stent where disintegration is desired to occur first. In this example, the lines are positioned on the outer diameter surface of every connector between strut rings. The lines are from one micron to 10 microns wide. Upon implantation, the physiological environment causes metal deterioration. The degradation (corrosion of magnesium) is accelerated by the presence of the chloride ions in the painted lines. The metal beneath the lines thins down first to a thickness where the applied stresses exceeds the load-bearing capability of the material thickness and fracture occurs. The stent is thereby broken into individual rings which subsequently degrade and disintegrate into small fragments and are eventually harmlessly bioabsorbed.

Other embodiments are in the claims.

What is claimed is:

1. An implantable medical endoprosthesis comprising a material, the implantable medical endoprosthesis having first and second regions, the first region comprising the material in a first solid phase, and the second region comprising the material in a second solid phase different from the first solid phase.

2. The implantable medical endoprosthesis of claim 1, wherein a length of the first region along a longitudinal axis of the implantable medical endoprosthesis is about 100 microns or more.

3. The implantable medical endoprosthesis of claim 1, wherein a length of the second region along a longitudinal axis of the implantable medical endoprosthesis is about 1 millimeter or less.

4. The implantable medical endoprosthesis of claim 1, wherein the material comprises magnesium and further comprises at least one member selected from the group consisting of iron, nickel, cobalt, and copper.

5. The implantable medical endoprosthesis of claim 1, wherein an erosion rate of the second region within a body lumen is larger than an erosion rate of the first region within the body lumen.

6. The implantable medical endoprosthesis of claim 1, wherein a thickness of the material in the second phase in a direction transverse to a longitudinal axis of the implantable medical endoprosthesis is about 25% or more of a maximum thickness of the first region in the same direction.

7. The implantable medical endoprosthesis of claim 1, comprising a plurality of alternating first and second regions having a common longitudinal axis.

8. The implantable medical endoprosthesis of claim 1, wherein a length of the first region along a longitudinal axis of the implantable medical endoprosthesis is about 100 microns or more.

9. The implantable medical endoprosthesis of claim 1, wherein a length of the first region along a longitudinal axis of the implantable medical endoprosthesis is about 1 millimeter or more.

10. The implantable medical endoprosthesis of claim 1, wherein a length of the second region along a longitudinal axis of the implantable medical endoprosthesis is about 1 millimeter or less.

11. The implantable medical endoprosthesis of claim 1, wherein a length of the second region along a longitudinal axis of the implantable medical endoprosthesis is about 100 microns or less.

12. A method of making an implantable medical endoprosthesis, the method comprising:
heating a region of an implantable medical endoprosthesis, the implantable medical endoprosthesis comprising a material, wherein heating the region of the implantable medical endoprosthesis converts the material from a first solid phase to a second solid phase different from the first solid phase; and
cooling the heated region under conditions that allow the material in the heated region to remain in the second phase.

13. An implantable medical endoprosthesis having first and second regions, the first region comprising a first material and the second region comprising the first material coated with a second material, the second material being selected to increase an erosion rate of the second region with respect to the first region in a body lumen.

14. The implantable medical endoprosthesis of claim 13, wherein the first material comprises magnesium.

15. The implantable medical endoprosthesis of claim 13, wherein the second material comprises at least one member selected from the group consisting of iron, nickel, cobalt, and copper.

16. The implantable medical endoprosthesis of claim 13, wherein the second material comprises an organic material.

17. The implantable medical endoprosthesis of claim 13, wherein the first region is coated with a third material to reduce an erosion rate of the first region relative to an uncoated region in a body lumen.

18. The implantable medical endoprosthesis of claim 13, wherein the second material forms a patterned coating on at least some surfaces of the second region.

19. An implantable medical endoprosthesis having first and second regions, the first and second regions comprising a material, and the second region having pores.

20. The implantable medical endoprosthesis of claim 19, wherein the second region of the implantable medical endoprosthesis has inner and outer surfaces, and at least some of the pores are located between the inner and outer surfaces of the second region.

21. The implantable medical endoprosthesis of claim 19, wherein the second region of the implantable medical endoprosthesis has inner and outer surfaces, and at least some of the pores are located at the inner or outer surfaces.

22. The implantable medical endoprosthesis of claim 19, wherein a thickness of the second region in a direction transverse to a longitudinal axis of the implantable medical endoprosthesis is less than a thickness of the first region in the same direction.

23. The implantable medical endoprosthesis of claim 19, wherein a length of the first region along a longitudinal axis of the implantable medical endoprosthesis is about 100 microns or more.

24. The implantable medical endoprosthesis of claim 19, wherein a length of the second region along a longitudinal axis of the implantable medical endoprosthesis is about 1 millimeter or less.

25. The implantable medical endoprosthesis of claim 19, wherein the material in the first region comprises pores.

26. The implantable medical endoprosthesis of claim 25, wherein the pores in the first region have a smaller mean diameter than a mean diameter of the pores in the second region.

27. The implantable medical endoprosthesis of claim 25, wherein the first region comprises fewer pores per unit volume than the second region.

28. A method of making an implantable medical endoprosthesis, the method comprising:
heating a region of an implantable medical endoprosthesis, the implantable medical endoprosthesis comprising a material, wherein the region of the implantable medical endoprosthesis is heated to a temperature greater than a melting temperature of the material;
disposing gas through the heated region; and
cooling the heated region so that at least some of the gas is trapped in the heated region.

29. A method of making an implantable medical endoprosthesis, the method comprising:
coating surfaces of a first region of an implantable medical endoprosthesis with a masking agent;
contacting a saline solution to be in contact with one or more surfaces of a second region of an implantable medical endoprosthesis;
directing an electric current to flow through the saline solution; and
removing the masking agent.

30. An implantable medical endoprosthesis having inner and outer surfaces that define a wall that extends along a longitudinal axis of the implantable medical endoprosthesis, a first region of the wall having a first thickness in a direction transverse to the longitudinal axis, and a second region of the wall having a second thickness in the same direction that is less than the first thickness, wherein the first and second regions comprise a material, and the second region has pores.

31. The implantable medical endoprosthesis of claim 30, wherein a length of the first region along the longitudinal axis of the implantable medical endoprosthesis is about 100 microns or more, and a length of the second region along the longitudinal axis of the implantable medical endoprosthesis is about 1 millimeter or less.

32. The implantable medical endoprosthesis of claim 30, wherein the implantable medical endoprosthesis comprises a plurality of alternating first and second regions having a common longitudinal axis.

33. An implantable medical endoprosthesis comprising a material, the implantable medical endoprosthesis having first and second regions, the first region comprising the material in a first solid phase, and the second region comprising the material in a second solid phase different from the first solid phase.

34. The implantable medical endoprosthesis of claim 33, wherein a length of the first region along a longitudinal axis of the implantable medical endoprosthesis is about 100 microns or more.

35. The implantable medical endoprosthesis of claim 33, wherein a length of the second region along a longitudinal axis of the implantable medical endoprosthesis is about 1 millimeter or less.

36. The implantable medical endoprosthesis of claim 33, wherein the material comprises magnesium and further comprises at least one member selected from the group consisting of iron, nickel, cobalt, and copper.

37. The implantable medical endoprosthesis of claim 33, wherein an erosion rate of the second region within a body lumen is larger than an erosion rate of the first region within the body lumen.

38. The implantable medical endoprosthesis of claim 33, wherein a thickness of the material in the second phase in a direction transverse to a longitudinal axis of the implantable medical endoprosthesis is about 25% or more of a maximum thickness of the first region in the same direction.

39. The implantable medical endoprosthesis of claim 33, wherein the material in the second phase is disposed adjacent to an outer surface of the first region.

40. The implantable medical endoprosthesis of claim 33, wherein the material in the second phase is disposed adjacent to an inner surface of the first region.

41. The implantable medical endoprosthesis of claim 33, comprising a plurality of alternating first and second regions having a common longitudinal axis.

42. A method of making an implantable medical endoprosthesis, the method comprising:
heating a region of an implantable medical endoprosthesis, the implantable medical endoprosthesis comprising a material, wherein heating the region of the implantable medical endoprosthesis converts the material from a first solid phase to a second solid phase different from the first solid phase; and
cooling the heated region under conditions that allow the material in the heated region to remain in the second phase.

43. An implantable medical endoprosthesis having first and second regions, the first region comprising a first material and the second region comprising the first material coated with a second material, the second material being selected to increase an erosion rate of the second region with respect to the first region in a body lumen.

44. The implantable medical endoprosthesis of claim 43, wherein the first material comprises magnesium.

45. The implantable medical endoprosthesis of claim 44, wherein the second material comprises at least one member selected from the group consisting of iron, nickel, cobalt, and copper.

46. The implantable medical endoprosthesis of claim 43, wherein the second material comprises an organic material.
